(19) United States
(12) Patent Application Publication
(10) Pub. No.: US 2010/0198052 A1
(43) Pub. Date: Aug. 5, 2010

(54) MRI-COMPATIBLE ARTICULATING ARMS AND RELATED SYSTEMS AND METHODS

(76) Inventors: Kimble Jenkins, Memphis, TN (US); Peter Piferi, Orange, CA (US)

Correspondence Address: MYERS BIGEL SIBLEY & SAJOVEC, PO BOX 37428, RALEIGH, NC, 27627, US

(21) Appl. No.: 12/694,865

(22) Filed: Jan. 27, 2010

Related U.S. Application Data
(60) Provisional application No. 61/147,860, filed on Jan. 28, 2009.

(51) Int. Cl. A61B 5/055; A61B 19/00

(52) U.S. Cl. 600/417

(57) ABSTRACT

The disclosure describes articulating arms that are supported directly or indirectly by MRI scanner beds for use during MRI-guided procedures.

### RELATED APPLICATION

This application claims priority to and the benefit of priority of U.S. Provisional Patent Application Ser. No. 61/147,860, filed Jan. 28, 2009, the contents of which are hereby incorporated by reference as if recited in full herein.

### FIELD OF THE INVENTION

The present invention relates generally to devices used during medical procedures and may be particularly suitable for use in MRI-guided procedures.

### BACKGROUND OF THE INVENTION

MRI guided interventional procedures are becoming more viable and may provide improved outcomes, alternative procedures and/or therapies over conventional imaging modalities and procedures.

### SUMMARY OF EMBODIMENTS OF THE INVENTION

Embodiments of the invention are directed to MRI compatible articulating arms that can be mounted to a scanner patient support surface or frame thereof. Embodiments of the invention support the arm(s) on a base, support surface and/or frame so that the arm(s) can translate with a patient in and out of a magnet bore for use in MRI-guided procedures.

Some embodiments are directed to medical tools. The tools include an MRI compatible articulating arm having opposing first and second ends. The first end is configured to be supported directly or indirectly by a scanner bed and the second end is configured to hold a device that remains over or on a patient over an entry site to a target intrabody location of a patient during an MRI guided procedure.

In some embodiments, tools also include a base member that is supported by the scanner bed and holds the articulating arm.

In some embodiments, the device is a mounting member with an aperture extending therethrough. The mounting member may be reside above or contact a patient's body. The mounting member may be configured to hold a trajectory guide frame held on an upper surface of the mounting plate so that an interventional surgical device can extend through the mounting plate aperture. The mounting member may be a mounting plate.

In some embodiments, the articulating arm includes at least two segments that are attached via a joint or joints that allow the arm to have a wide range of motion. The articulating arm can be formed of non-ferromagnetic material.

The articulating arm may include a plurality of serially connected segments with at least one of the segments divided into portions that can rotate relative to each other and selectively lock into position.

In some embodiments, the tool held by the second end of the arm includes at least one surface coil and/or at least one gradient coil.

The mounting member can be held in a package for maintaining a sterile condition prior to surgical use.

Other embodiments are directed to MRI surgical systems. The systems include: (a) an articulating arm having one end portion that is supported (directly or indirectly) by a scanner bed and having an opposing end portion with a mounting member; and (b) a trajectory guide frame that is attachable to the mounting member.

The arm or portions thereof can be sterilized or covered with a sterile drape, sheath or sleeve for medical use.

The mounting member can include a through-port and the mounting member can be configured to reside on or above a patient's body so that the through-port resides over a target intrabody surgical entry site during an MRI procedure.

Still other embodiments are directed to surgical systems that include: (a) a patch adapted to reside on a patient, the patch having an MRI visible grid; (b) an MRI compatible articulating arm; (c) a trajectory frame attached to or configured to attach to the arm; and (d) a processor for defining an entry site to an intrabody target in the patient using the grid.

The system can include a clinician workstation having a display in communication with the processor and an MRI scanner.

Yet other embodiments are directed to methods for accessing a target intrabody surgical site for an MRI guided surgical procedure. The methods include: (a) placing an articulating arm (directly or indirectly) on a scanner bed; (b) adjusting the articulating arm to position a mounting member attached to the articulating arm over a desired entry site to a target intrabody surgical location; and (c) performing an MRI guided procedure while the mounting member is held by the articulating arm over the entry site.

Optionally, the mounting member can be a mounting plate and the methods may also include: placing the mounting plate on a patient's body; securing a trajectory guide frame to the mounting plate on an upper surface thereof; and directing a surgical tool to extend into the patient's body through an aperture in the mounting plate during the MRI guided procedure.

In some embodiments, the mounting member includes a surface coil in communication with an MRI scanner. The method can further include transmitting RF signal and/or obtaining MRI signal data using the mounting member surface coil during the MRI guided procedure.

Other embodiments are directed to medical kits for an MRI guided procedure. The kits include a plurality of discrete segments with hinge joints that are attachable in situ to form a non-ferromagnetic articulating arm.

One of the segments in the kit may optionally include a mounting member for holding a trajectory guide frame thereon.

Yet other embodiments are directed to base members for an MRI guided medical procedure. The base member is non-ferromagnetic and has a plurality of predefined anchor locations thereon for releasably attaching an articulating arm thereto. The base member is configured to reside on a scanner bed and translate with the patient in and out of a magnet bore.

Further features, advantages and details of the present invention will be appreciated by those of ordinary skill in the art from a reading of the figures and the detailed description of the preferred embodiments that follow, such description being merely illustrative of the present invention. Features described with respect with one embodiment can be incorporated with or into other embodiments although not specifically discussed therewith. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail in the specification set forth below.

### BRIEF DESCRIPTION OF THE DRAWINGS

FIG. 1A is a schematic illustration of an MRI compatible articulating arm supported by a scanner bed according to embodiments of the present invention.

FIG. 1B is a schematic illustration of an MRI compatible articulating arm attached to a base member residing on the scanner bed according to embodiments of the present invention.

FIG. 1C is a schematic illustration of an MRI compatible articulating arm attached to a base member according to embodiments of the present invention.

FIG. 2A is a schematic illustration of an MRI compatible articulating arm having interchangeable end attachment members according to embodiments of the present invention.

FIG. 2B is a schematic illustration of a set of components that can be used to configure an articulating arm according to embodiments of the present invention.

FIG. 2C is a schematic illustration of an alternative set of members for a single-use disposable mounting member according to embodiments of the present invention.

FIG. 3A is a partial top perspective view of an MRI compatible arm illustrating an end with a mounting member attached thereto according to embodiments of the present invention.

FIG. 3B is a partial top perspective view of an MRI compatible arm illustrating an end with an alternatively configured mounting member attached thereto according to embodiments of the present invention.

FIG. 3C is a partial top perspective view of an MRI compatible arm illustrating an end with another alternatively configured mounting member attached thereto according to embodiments of the present invention.

FIG. 3D is a partial top perspective view of an MRI compatible arm illustrating an end with yet another alternatively configured mounting member attached thereto according to embodiments of the present invention.

FIG. 3E is a partial bottom perspective view of another exemplary mounting member configured according to embodiments of the present invention.

FIG. 4A is a schematic illustration of a mounting member with a trajectory guide frame mounted thereon according to some embodiments of the present invention.

FIG. 4B is a side perspective illustration of an exemplary trajectory guide assembly according to embodiments of the present invention.

FIGS. 5A and 5B are top view schematic illustrations of an articulating arm with a mounting member that can be used with a scanner bed to carry out an MRI guided procedure at different sites according to embodiments of the present invention.

FIG. 6A is a side perspective view of an articulating arm with the mounting member positioned over a desired target entry site aligned with grid coordinates according to embodiments of the present invention.

FIG. 6B is a side view of an exemplary grid that can define target entry coordinates and/or point according to some embodiments of the present invention.

FIG. 7 is an end view of the articulating arm in an exemplary position in a scanner bore and attached to a patient support surface according to embodiments of the present invention.

FIG. 8 is an end view of the articulating arm shown in FIG. 7 illustrated in an alternative configuration and position in a scanner bore according to embodiments of the present invention.

FIG. 9 is an enlarged top view of the articulating arm shown in FIG. 7 according to embodiments of the present invention.

FIG. 10 is an end view of the articulating arm shown in the position illustrated in FIG. 9.

FIG. 11 is an enlarged end view of the articulating arm and configuration thereof shown in FIG. 9.

FIG. 12 is an end view of an articulating arm and base member according to embodiments of the present invention.

FIG. 13 is a partial side view of a base member with an anchor member being on a scanner bed used for an MRI-guided procedures, shown with a torso target entry site according to embodiments of the present invention.

FIG. 14 is a partial top perspective view of the articulating arm used for a back entry site (e.g., for a spinal or other target intrabody site accessed from a back side of the patient) according to embodiments of the present invention.

FIG. 15 is a flow chart of steps that can be used to carry out embodiments of the present invention.

FIG. 16 is a block diagram of a system according to embodiments of the present invention.

FIG. 17 is a screen shot of an exemplary UI (User Interface) and visualization on a display according to embodiments of the present invention.

FIG. 18 is a block diagram of a data processing system according to embodiments of the present invention

### DETAILED DESCRIPTION OF EMBODIMENTS OF THE INVENTION

The present invention now is described more fully hereinafter with reference to the accompanying drawings, in which some embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

Like numbers refer to like elements throughout. In the figures, the thickness of certain lines, layers, components, elements or features may be exaggerated for clarity.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on”, “attached” to, “connected” to, “coupled” with, “contacting”, “supported by” etc., another element, it can be directly on, attached to, connected to, coupled with or contacting the other element or intervening elements may also be present (e.g., indirectly supported, attached, coupled, contacting, connected, coupled, etc. . . . ). In contrast, when an element is referred to as being, for example, “directly on”, “directly attached” to, “directly connected” to, “directly coupled” with, “directly supported by” or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is inverted, elements described as “under” or “beneath” other elements or features would then be oriented “over” the other elements or features. Thus, the exemplary term “under” can encompass both an orientation of “over” and “under”. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

The term “scanner bed” refers to a patient support surface or frame thereof (which is typically relatively rigid) that, in operative position, resides in a region of a homogeneous high magnetic field associated with a Magnetic Resonance Imaging (MRI) scanner during active image signal acquisition. The scanner bed can typically translate in a longitudinal direction to position the patient in the homogeneous magnetic field region of the magnet. MRI scanners are well known to those of skill in the art and include, for example, the SIGNA 1.5T/3.0T from GE Healthcare: the ACHEIVA 1.5T/3.0T and the INTEGRA 1.5T from Philips Medical System; and the MAGNETOM Avanto, the MAGNETOM Espree, the MAGNETOM Symphony, and the MAGNETOM Trio, from Siemens Medical.

The term “MRI-compatible” means that a device is safe for use in an MRI environment that can operate as intended in an MRI environment and not introduce artifacts into MRI images. As such, if residing within the high-magnetic field region of the magnet, the MRI-compatible device is typically made of a non-ferromagnetic material(s) suitable to reside and/or operate in a high magnetic field environment. The term “high magnetic field” refers to magnetic fields above 0.5 T, typically between 1.5 T to 10 T. The term “tool” refers to devices that facilitate medical procedures. Embodiments of the invention are particularly suitable for veterinarian or human therapeutic or diagnostic use, but may be used for research or other purposes. The term “sterile” and derivatives thereof means that the component meets regulatory clinical cleanliness standards for medical procedures. The term “wide range of motion” refers to arms that can be configured into many different shapes to reside over desired anatomical entry sites into the body.

The term “fiducial marker” refers to a marker that can be electronically identified using image recognition and/or electronic interrogation, typically interrogation of MRI image data. The fiducial marker can be provided in any suitable manner, such as, but not limited to, a geometric shape, a component on or in the device, a coating or fluid-filled component or feature (or combinations of different types of fiducial markers) that makes the fiducial marker(s) MRI-visible with sufficient signal intensity (brightness) for identifying location and/or orientation information for the device and/or components thereof in space.

Embodiments of the present invention can be configured (and/or used) to carry out or facilitate MRI guided procedures, including, for example diagnostic and interventional procedures such as to guide and/or place interventional devices to any desired internal region of the body or object, including deep brain sites for neurosurgeries or other target intrabody locations for other procedures. The object can be any object, and may be particularly suitable for animal and/or human subjects. For example, the system and/or devices thereof can be used for gene, e.g., antibody, and/or stem-cell based therapy delivery or other therapy delivery to intrabody targets in the brain, heart, lungs, liver, kidney, ovary, stomach, intestine, colon, spine or to other locations. In addition, embodiments of the systems can be used to treat cancer sites. In some embodiments, the systems can be used to ablate tissue and/or deliver pharmacologic material in the brain, heart or other locations. In some embodiments, it is contemplated that the systems can be configured/used to treat AFIB, deliver stem cells or other cardio-rebuilding cells or products into cardiac tissue, such as a heart wall, via a minimally invasive MRI guided procedure while the heart is beating (i.e., not requiring a non-beating heart with the patient on a heart-lung machine). Embodiments of the present invention may be particularly suitable for use in MRI procedures that have soft tissue intrabody entry sites which lack sufficient structural rigidity to either support a tool in a desired position and/or to maintain a defined trajectory path to the intrabody target site.

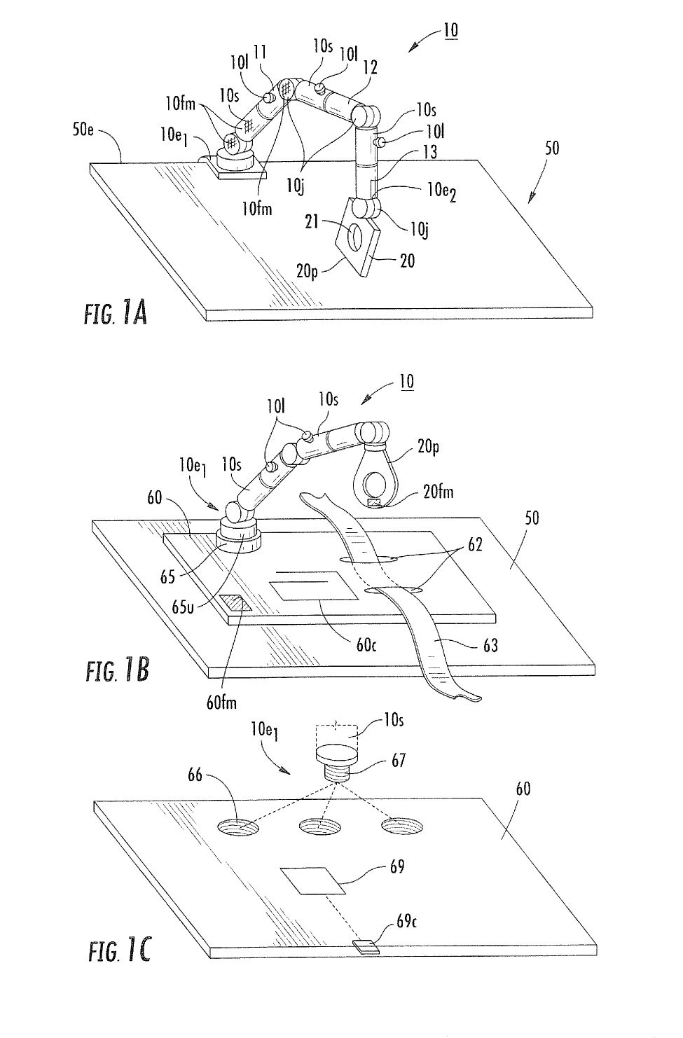

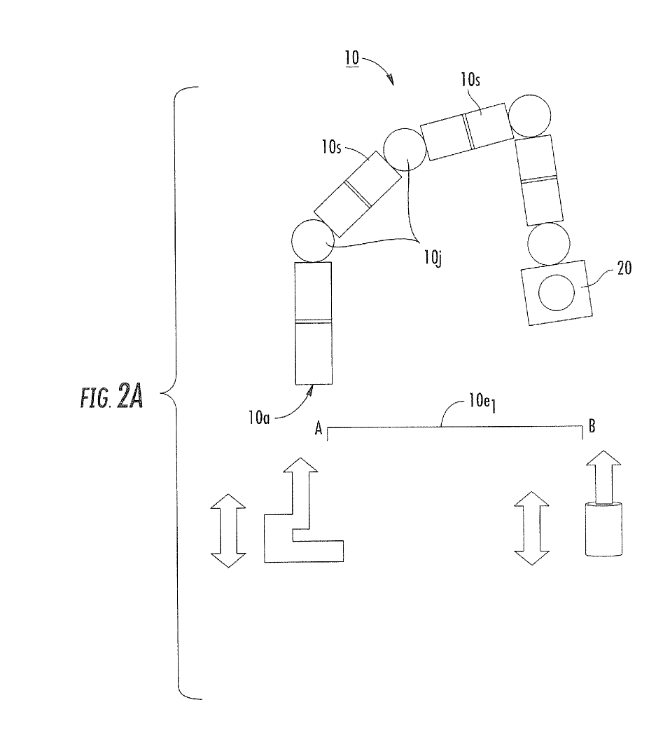

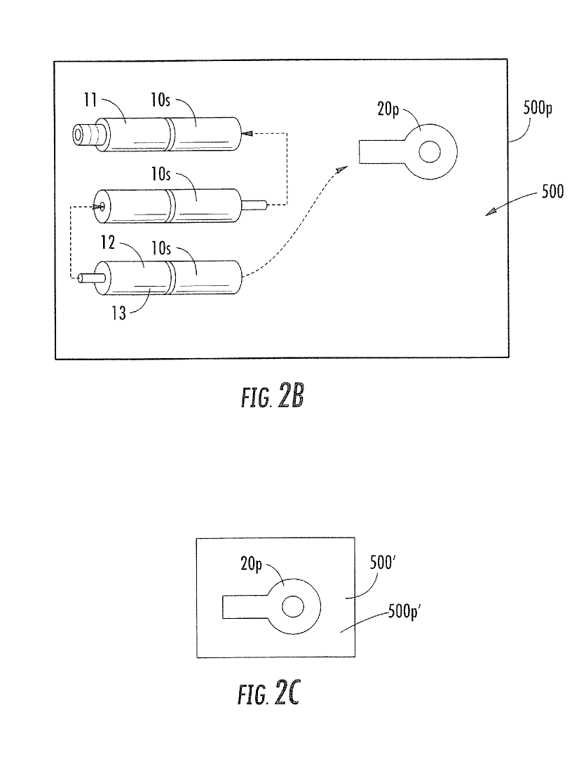

Referring now to the figures, FIG. 1A illustrates an example of an articulating arm 10 having one end portion 10e₁ supported by a scanner bed 50 of an MRI scanner. The other opposing end portion of the arm 10e₂ can be attached to a surgical tool 20 such as a mounting plate, mounting basket, box, or other component or tool. In the embodiment shown, the surgical tool 20 is a mounting member 20p. The mounting member 20p can be a mounting plate that is substantially planar and has a through-aperture 21 through which interventional tools can access an intrabody target site using a planned trajectory. The aperture 21 can be circular or in any desired shape and may be in the form of a recess or slot in the plate with an open side rather than having a closed perimeter (FIG. 3D). The mounting member 20 can have a body with sidewalls (open or closed) (FIG. 3E). The tool 20 (e.g., mounting member such as a plate 20p) can reside on or above a patient, typically residing on an outer surface of a patient or a surface that is made visible for the surgical procedure (FIGS. 5A, 5B). In other embodiments, the tool 20 may attach to the patient such as via adhesives or other attachment means, typically aligned with the intrabody target surgical site.

The arm 10 is an articulating arm that rises and can extend across the scanner bed 50. The arm 10 can be self-supporting and able to retain a desired configuration as it is placed in different configurations. The arm 10 can have multiple degrees of freedom with joints 10j connecting a plurality of arm segments 10s that can allow a wide, even full, range of motion in three dimensions, e.g., X, Y and Z axes, and can be fixed in a desired configuration when tightened and/or locked. The hinge joints 10j that connect the different segments 10s can be knuckle hinge joints, pivot joints, saddle joints and ellipsoid joints, for example. The hinge joints 10j can be the same or different for each segment. The arm 10 can include lockable rotation adjustment members 10l (shown as thumbscrews in FIG. 1A but other configurations may be used such as depressible self or manual locking members or buttons). The rotational adjustment allowed by the segments and the angular adjustment of the hinges allow for a substantially full range of motion. The arm 10 can also include at least one, typically a plurality of spaced apart, fiducial markers 10fm. The MRI-guided system can be configured to electronically determine the position and/or orientation of the arm 10 using the fiducial markers 10fm. The mounting member 20p and/or base 60 can also include respective fiducial markers 20fm, 60fm (FIG. 1A) and the system can be configured to determine orientation, location or position of a desired component or device using data from one or more of the respective markers 10fm, 20fm, 60fm.

The arm 10 can be sized and configured to fit within the bounds of a bore of a magnet (for closed bore systems). The end of the arm 10e₁ supported by the scanner bed 50 can attach directly or indirectly to the scanner bed, e.g., attach to a top of the scanner bed, a side edge of the scanner bed 50e (FIG. 1A), or a base member 60 residing on scanner bed 50 (FIG. 1B, 7). The arm 10 can translate in and out of the magnet bore with the patient and scanner bed 50 and remain in a fixed position relative to the patient.

The arm 10 can be attached to the base member 60 or scanner bed 50 using any suitable, typically releasable attachment, including a vise, clamp, screw, bayonet insert, pin, vacuum, adhesive, VELCRO, mating or threaded channel or combinations thereof or any other type of attachment that provides a sufficiently secure attachment.

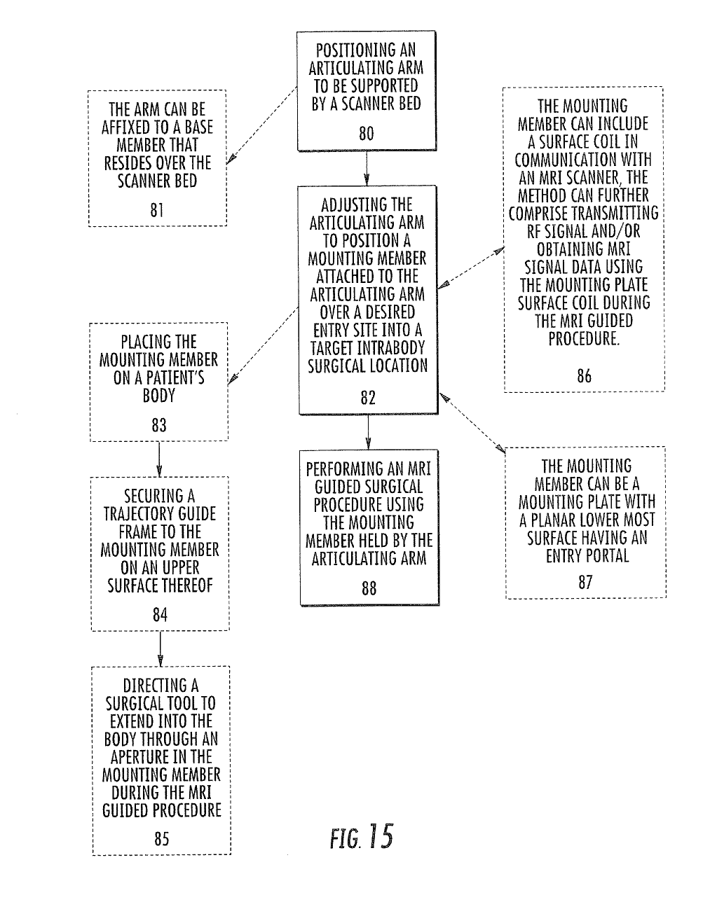

FIG. 1B illustrates that the base member 60, which typically resides under a patient during use and is MRI compatible, can include an optional surface coil 60c that communicates with the MRI scanner for providing image data. The base member 60 can have the same or a smaller width than the underlying scanner bed 50 or may have a smaller width. If the base member 60 has a greater width it should be configured to not interfere with the translation of the bed into the scanner bore. The length of the base member 60 can be the same, longer or shorter than the scanner bed 50 but is typically shorter as shown in FIG. 1B. A sterile sheet 66 (FIG. 13) can be placed over the base member 60 for patient comfort or sterility. The base member 60 can be sterilized prior to use or the sheet or surgical drape can provide the sterile barrier where needed.

The base member 60 can be a substantially rigid non-ferromagnetic member and may also include patient and/or arm fixation means, such as slots 62 for fixation straps 63 to hold a patient 100 in position and/or the base to the scanner bed 50. The base member 60 can reside without positive retention components on the scanner bed 50. In other embodiments the base member 60 can be affixed to the scanner bed 50 via any suitable means, including, for example, slots formed into the upper surface of the scanner bed that receive projections formed on a lower surface of the base member, straps, side clamps, VELCRO, adhesives, and the like. The patient can also be secured to the base to immobilize the patient relative to the arm 10 and/or base 60 using any suitable means including straps, clamps, VELCRO, frames with screws (e.g., head or body fixation frames).

In some embodiments, the base member 60 can include at least one integral anchor 65 in one or more pre-defined locations that can form or cooperate with another member to form the mounting attachment mechanism to releasably attach the arm 10. In some embodiments, the base anchor 65 can include hardware that attaches to an end 10e₁ of the arm 10 or the base member can define the mounting attachment.

As shown in FIG. 1C, the base member 60 can include a female threaded aperture 66 that threadably receives a male screw 67 on an end of a segment 10s of the arm 10. The female/male configurations can also be reversed. In other embodiments, the anchor 65 can include an upwardly extending member 65u that resides on the base member 60 and releasably attaches to an end of the arm 10e₁ (FIG. 1A). The member 65u can be attached to the base prior to or during the medical procedure before the remainder of the arm 10 is attached. In some embodiments, the member 65u can be pre-formed into the base member 60 (such as by an OEM or prior to placing the base member 60 on the scanner bed 50). Other base member 60 and arm 10 attachment configurations may be used as discussed above with respect to the examples of attachments.

In some embodiments, a user can select to attach the arm 10 to different (pre-defined or pre-shaped) locations on the base member 60 according to the target surgical site or patient shape/anatomy. In some embodiments, as also shown in FIG. 1C, the anchor(s) 65 (shown as a plurality predefined different anchor locations that can each selectively attach to the arm) are formed on one side of the base member 60 and the base member 60 can be flipped if a user desires to use the arm mount(s) 65 on the other side.

FIG. 1C also illustrates that the base member 60 can hold or include a grounding pad/electrode 69. Such a grounding pad 69 can cooperate with electrodes associated with a defibrillator that can be used during some surgical procedures, such as, for example, cardiac electrophysiology procedures, as is known to those of skill in the art. The grounding pad 69 can be used with the slots 62 and/or RE coil shown in other figures. The grounding pad 69 can optionally include an electrical lead that is integral to and/or supported by the base 60 and optionally can include a connector 69c on a perimeter as shown. In other embodiments, the grounding pad 69 does not require an electrical lead or connector. In yet other embodiments, the grounding pad 69 can include a proximately located connector (residing inside the perimeter) and/or a lead can be provided to connect the pad to electrical ground and/or a defibrillator as desired.

The arm 10 and anchor 65, where used, can also be located along the ends (short sides) rather than a long side/side portion or in addition to a long side of the scanner bed 50 and/or base member 60.

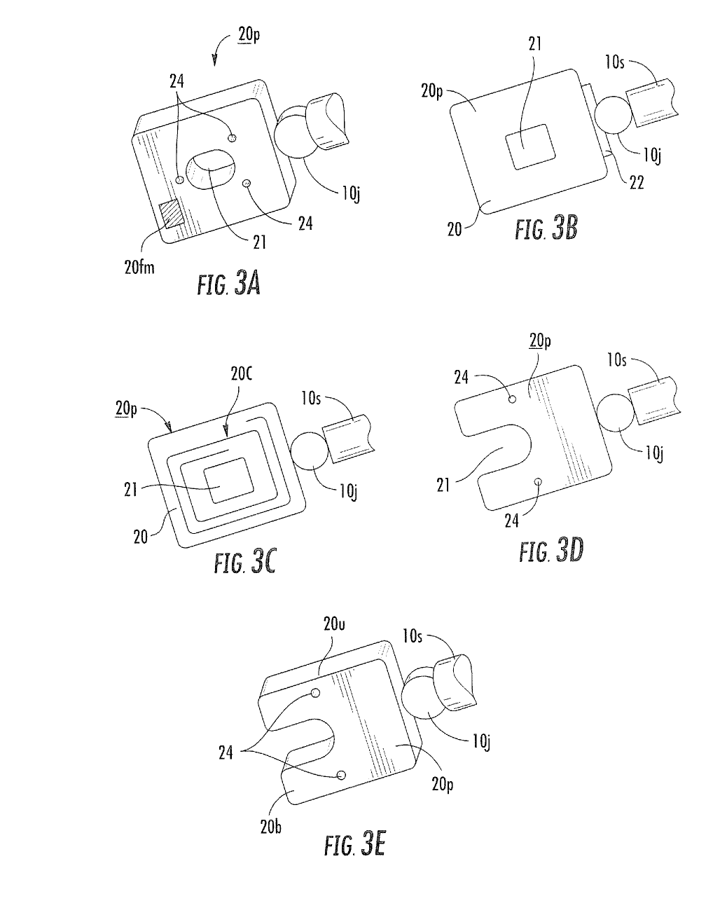

FIG. 2A illustrates that the arm 10 can have a modular attachment end 10a that can accept alternatively configured scanner bed mounting hardware A, B, each with a different attachment configuration to allow a user to select the appropriate configuration for the scanner bed 50 in use. A user can order the end A/B desired for use with the arm 10, or a surgical kit can include both or A or B ends 10e₁. In still other embodiments, a surgical tool kit or package can include the correct end 10e₁ (A, B) with the other portion(s) of the arm 10 included depending on the part number ordered (the part number being correlated to scanner type and matable attachment end with the connection/attachment type for the scanner to be used). Additionally, two or more of the segments 10s can be pre-attached together. Alternatively, all or some of the segments 10s can be provided separately for onsite assembly as discussed below.

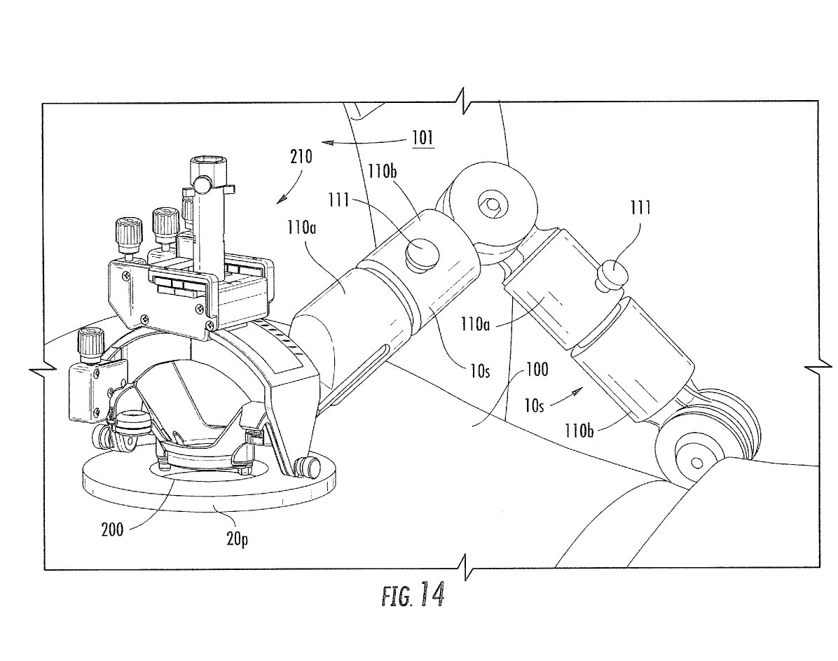

As shown in FIG. 2B, one or all of the segments 10s can be provided in a set such as in a medical kit 500 for onsite assembly by a user. The kit 500 can hold the one or more segments 10s in a sterile package 500p and provided for assembly by a user to customize a particular medical use. The segments 10s can be provided in different lengths and/or the same lengths and selectively attached to each other to form the desired length or configuration (FIG. 13 shows the arm with two segments, FIG. 1A with three segments, and FIG. 11 with four segments). As such, one or more of the segments 10s and joints 10j may be matably attached in situ by a user without requiring special tooling (or indeed any tooling). In some embodiments, one of the segments 10s (referenced as segment 11 in the kit 500) can be configured with an end mount configuration 10e₁ that may be different from other segments. The mounting member 20p can be provided as an integral part of one link segment 10s or may matably attach to a link segment. The kit may be configured to provide additional segments to an onsite mounting member 20p and/or anchor portion to allow a user to customize the arm 10 for a particular patient or procedure.

Referring to the embodiment shown in FIG. 2C some of the segments 10s of the arm, e.g., typically all but the segment 10s with the mounting member 20p, may be reusable and sterilized by a medical facility prior to a procedure. The mounting member 20p (where a separate component) or the segment 10s integrated with the mounting member 20p (where an integral component thereof) can be provided in a kit 500′ in sterile package 500p′ for attachment to the remainder of the arm onsite as this portion of the device may be single-use disposable.

As shown in FIG. 1A, the arm 10 has at least two, shown as three, serially connected articulating segments 10s, e.g., segments 11, 12, 13. The segments 10s can be provided in two or more than three segments as well. FIG. 7 illustrates four segments 10s, numbered as elements 11-14. FIG. 12 illustrates two segments 10s. The segments 10s can have the same or different lengths and the same or different configurations (e.g., cylindrical). The arm 10 (and segments thereof) can be formed from any suitable material, typically a light-weight relatively rigid polymeric material, such as, for example, fiberglass, ceramics, fiber reinforced resins, PEEK, ABS, polycarbonate, KEVLAR, Garolite. However, non-magnetic metals or other materials may also be used.

The arm 10 may be particularly suitable for use in MRI-guided procedures where the procedure is carried out in an MRI scanner or MRI interventional suite. The arm 10 may be useful for many different procedures as discussed above, e.g., deep brain procedures, spinal procedures, cardiac procedures, including but not limited to, cardiac EP procedures where heat or cryogenic ablation is used, as well as intrabody biopsies or treatment of any target organ or tissue, including breast, liver, thyroid, lung, kidney, ovarian, cervical, prostate, urethra, colon, intestine, stomach, and the like. The arms 10 may be suitable for MRI-guided procedures that deliver therapeutic agents, such as antigen, antibody and/or gene therapies, stem cells and the like.

The arm 10 can be sterilized and may optionally be single-use disposable or portions thereof may be single-use disposable as discussed above. Alternatively, or additionally, a sterile cover or case can be used to cover the arm 10 or at least a major portion thereof during the procedure as appropriate. The arm 10 can be a “universal” arm configured for multiple different procedures or may be procedure-specific, e.g., a spinal arm, a cardiac arm, a torso arm, a leg, arm, breast, prostate, colon, etc. . . . The arm 10 and/or base member 60 can also be “universal” in that one or both can be used interchangeably with different MRI scanner systems from different scanner manufacturers. Alternatively, the base member 60 and/or the arm 10 may be scanner or scanner manufacturer specific.

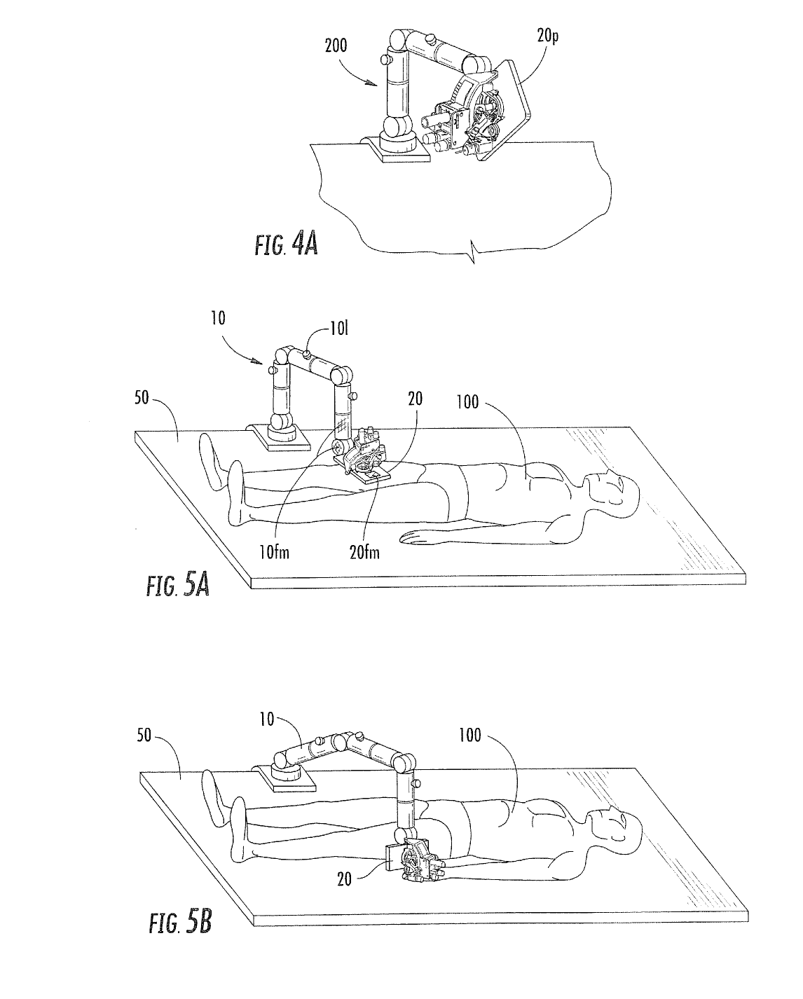

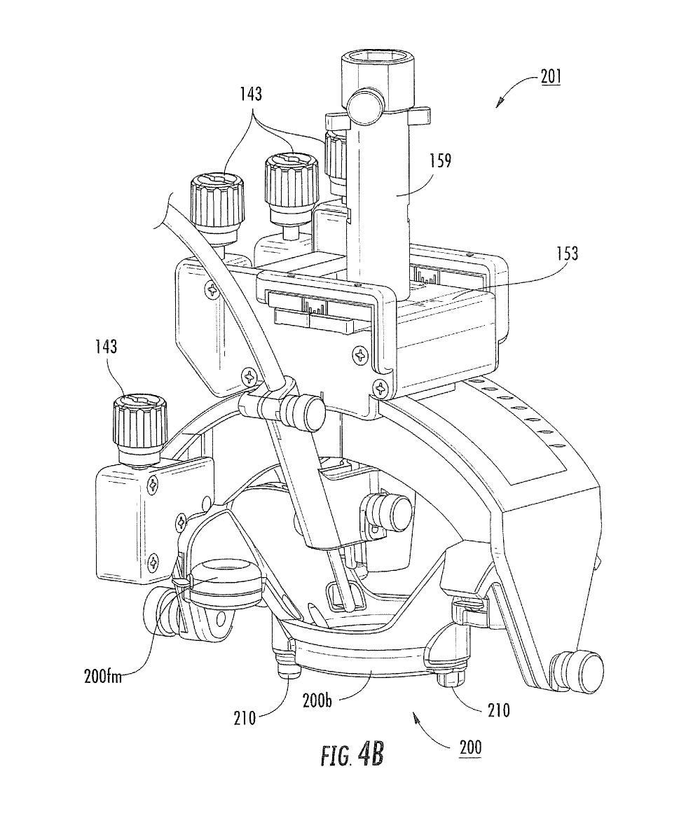

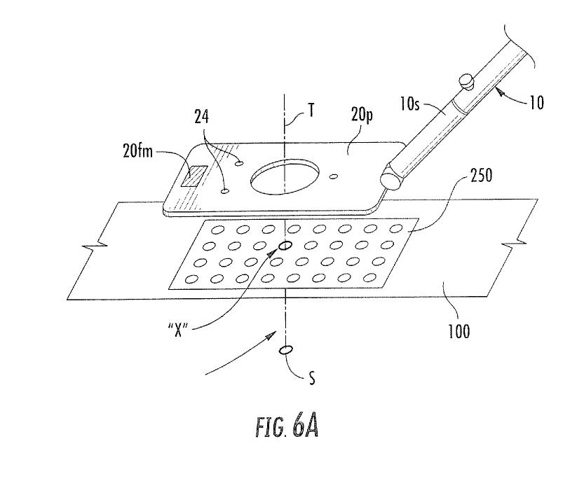

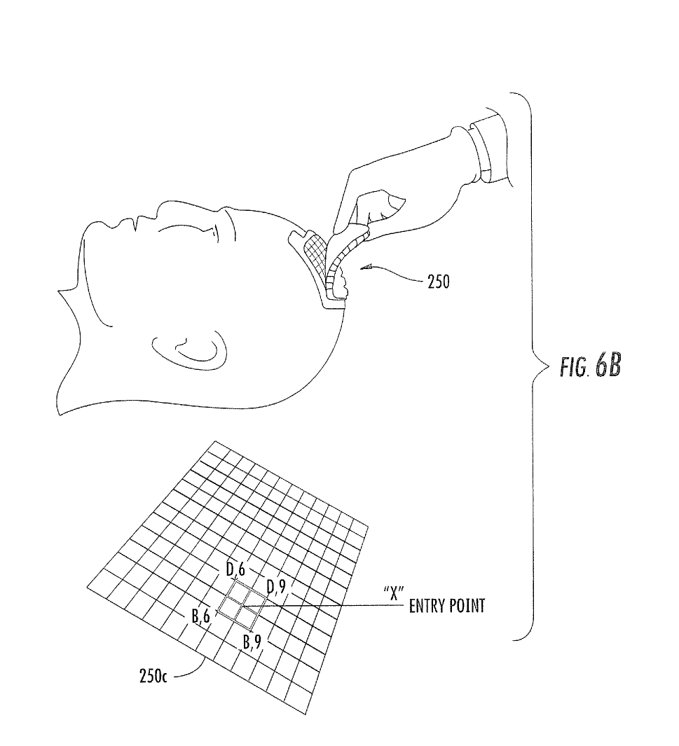

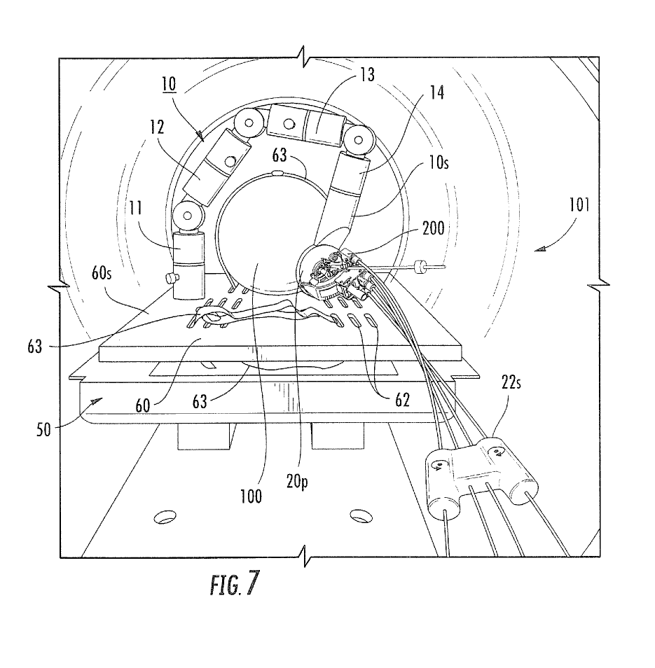

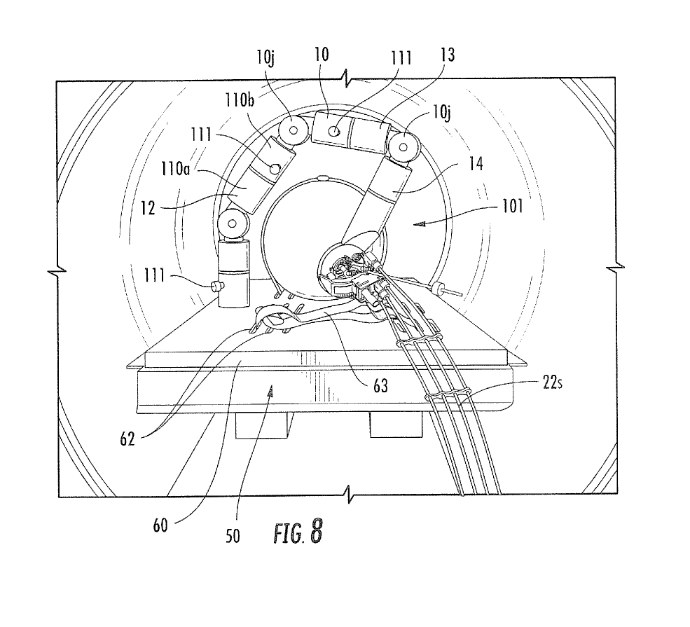

FIG. 3A illustrates an example of a tool 20 that can be attached to the end portion of the arm 10. In this embodiment, the tool 20 can include a mounting member 20p and can include a through-aperture 21 (e.g., a portal) and mounting apertures 24 spaced apart about the perimeter of the aperture 21 that can be used to accept fixation members for releasably attaching a trajectory guide frame 200 (FIGS. 4A, 4B, 6A). The fixation members can include but are not limited to pins, screws, staples, nails, or other fixation devices. The mounting member 20p can include a joint 10j on an outer perimeter thereof that connects to an articulating arm segment 10s. FIG. 3B illustrates that the mounting member 20p can have an extension 22 that resides between the mounting member/plate 20p that merges into or is fixedly attached to the hinge or joint 10j. Alternatively, the plate 20p can be integral to one of the segments 10s. The aperture 21 may be substantially centered or offset relative to a center of the plate 20p. FIG. 3D illustrates that the mounting member 20p can have a recess or slot that provides the aperture or access port 21. FIG. 3E illustrates that the mounting member 20p can have upwardly extending sidewalls 20s (e.g., extending upwardly from the bottom surface 20b).

FIG. 3C illustrates a mounting member 20p that may be particularly useful for MRI procedures, with the mounting member 20p optionally including one or more coils 20c, such as one or more gradient coils and/or one or more surface coils. The coil(s) 20c can communicate with the MRI scanner. If a surface coil, coil 20c can be in communication with the MRI scanner and configured to transmit and/or receive MRI signals to generate high resolution images.

FIG. 4A illustrates that in some embodiments, a trajectory guide frame 200 can be securely attached to an outer surface of the mounting member 20p. FIG. 4B illustrates an example of a trajectory guide assembly 201 (although other trajectory guide assemblies 201 and frames 200 may also be used). The arm 10 can facilitate the secure placement of the mounting member 20p (and, where used, frame 200) directly over and aligned with the planned site of entry into the body for the procedure. Where used, the trajectory guide frame 200 can be attached to the plate 20p before or after the plate 20p is locked into the desired operative position, typically attached to the patient. The trajectory guide frame 200 can be used to define and hold a trajectory path into the body to reach the target site. The trajectory guide frame 200 may be used with planning software to provide a controlled and precise trajectory for a device to an intrabody target.

In some embodiments, a user can place the arm on the scanner bed 50 and/or base member 60 and position the mounting member 20p over the patient during the planning stage. The arm 10 can be locked into the desired configuration and position relative to the patient, then a portion or segment 10s (typically the one with the mounting member 20p) disconnected temporarily while the frame 200 (and assembly 201, where used) is attached to the mounting member 20p. The segment can then be reconnected to continue the procedure. A user may optionally be translated out of the bore for the disconnecting/attaching frame/reconnecting steps, then translated back in.

FIG. 4B illustrates that the frame 200 can hold a trajectory assembly 201 that includes a platform 153 for X-Y adjustment, and actuators 143 that can adjust the pitch, roll and X-Y adjustments of the trajectory. The frame 200 can hold a targeting canula 159. The frame 200 can include a base 200b with screws or outer mounting members 210 that can attach to the mounting member 20p (such as to apertures 24, FIG. 3A). The frame 200 can include at least one fiducial marker 200fm. For additional discussion of suitable trajectory guides, see, U.S. application Ser. No. 12/134,412, and co-pending, co-assigned U.S. patent application Ser. No. 12/236,950, the contents of which are hereby incorporated by reference as if recited in full herein.

FIGS. 5A and 5B illustrate that the arm 10 can be configured so that it can be used for different surgical sites, shown as mounted to a left side of the bed 50 to be able to extend a distance so as to reach both left and right side target locations of the patient 100. However, the arm 10 can mount to either side of the bed 50 and may be configured to extend only partially across the patient 100 and or scanner bed 50. The arm 10 can be customized to fit different patients and/or target sites using releasably attachable segments 10s. Thus, the arm 10 can be adjusted in situ during or prior to a surgery for a particular patient. In some embodiments, the arm 10 can have a common base (anchor) structure and the intermediate portion can be extended or shortened using additional or lesser numbers of segments 10s as appropriate for a particular patient or procedure. Similarly, an end portion can be interchangeably modified with different tools (e.g., mounting devices) to accommodate different procedures.

FIG. 6A illustrates that placement of the mounting member 20p or other tool 20 can be facilitated by the use of an MR visible marking grid 250 placed on the body of the patient 100. The marking grid 250 can be used with automated planning software to help define the appropriate trajectory path “T” and target site “S” in the body. The grid 250 can indicate the appropriate coordinates 250c (FIG. 6B) that provide the desired entry point “X” into the body based on the marking grid 250 and the automated software. FIG. 13 also illustrates the use of a marking grid 250.

The term “grid” refers to a pattern of crossed lines or shapes used as a reference for locating points or small spaces, e.g., a series of rows and intersecting columns, such as horizontal rows and vertical columns (but orientations other than vertical and horizontal can also be used). The grid can include at least one fiducial marker. The grid can include associated visual indicia such as alphabetical markings (e.g., A-Z and the like) for rows and numbers for columns (e.g., 1-10) or the reverse. Other marking indicia may also be used. The grid can be provided as a flexible patch that can be releasably attached to the skin of a patient. For additional description of suitable grid devices, see co-pending, co-assigned U.S. patent application Ser. No. 12/236,621, the contents of which are hereby incorporated by reference as if recited in full herein.

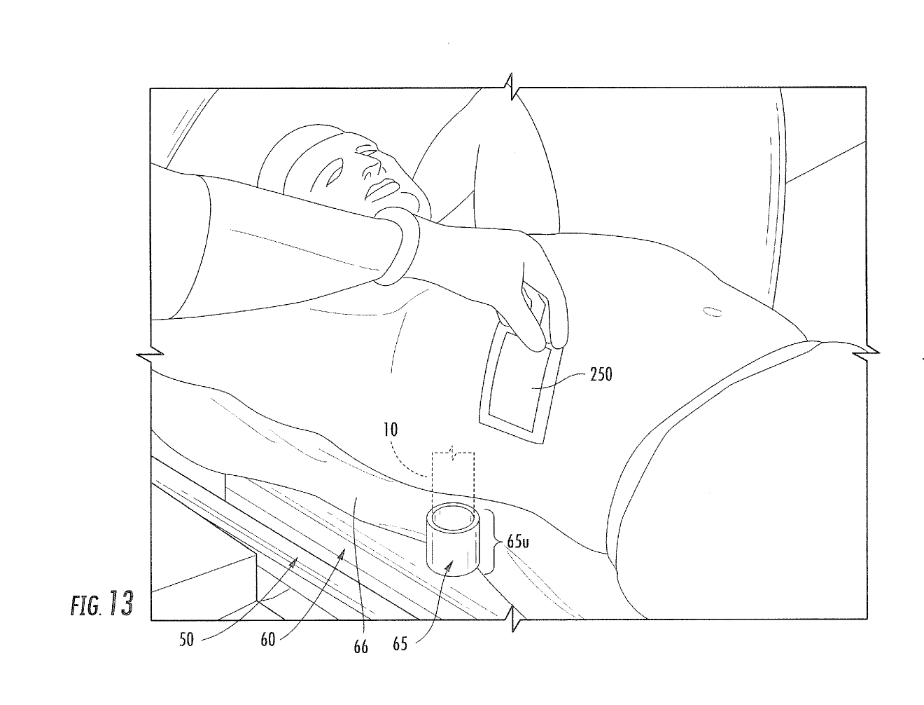

FIGS. 7-11 illustrate an arm 10 attached to an upper surface 60s of the base member 60, with the base member 60 supported by the scanner bed 50. As shown, the arm 10 has four articulating segments 10s (identified as segments 11-14). A trajectory guide frame 200 is mounted to the plate 20p. A hand controller 225 can be attached to actuators 143 of the assembly 210 that translate a trajectory canula 159 that is held by the frame 200 to adjust the trajectory (pitch, roll, X-Y movement). It is noted that FIGS. 7-11 and 14 illustrate one embodiment of the arm 10 and the mounting plate 20p (and scanner attachment configuration and trajectory guide) but the arm 10 is not limited to this configuration nor is the mounting plate 20p and other configurations for each may be used as well as alternative mounting arrangements and trajectory guides or tool supports.

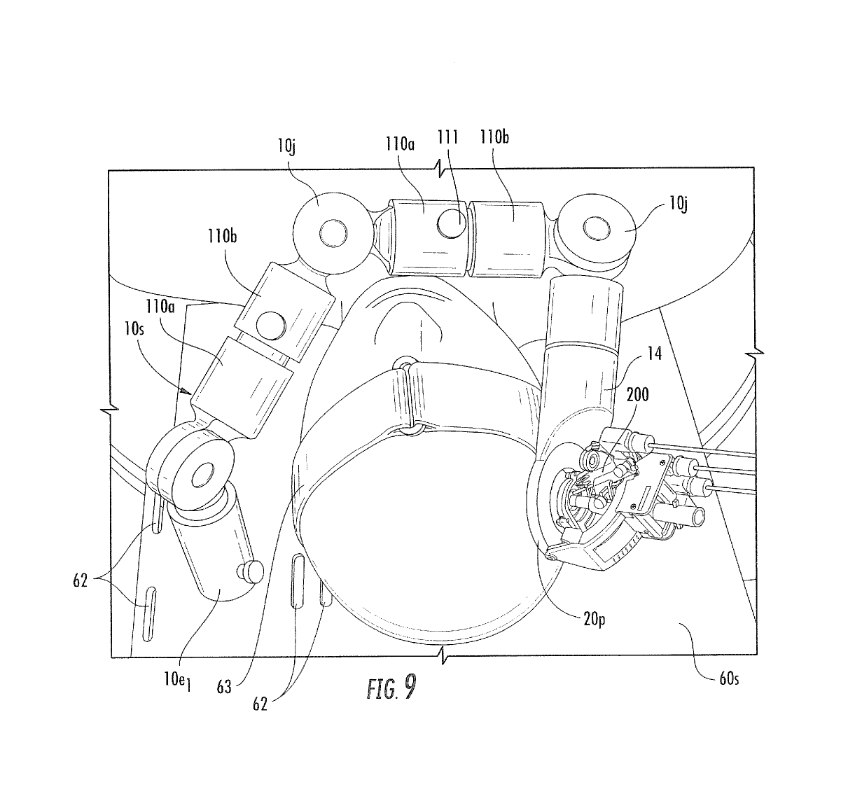

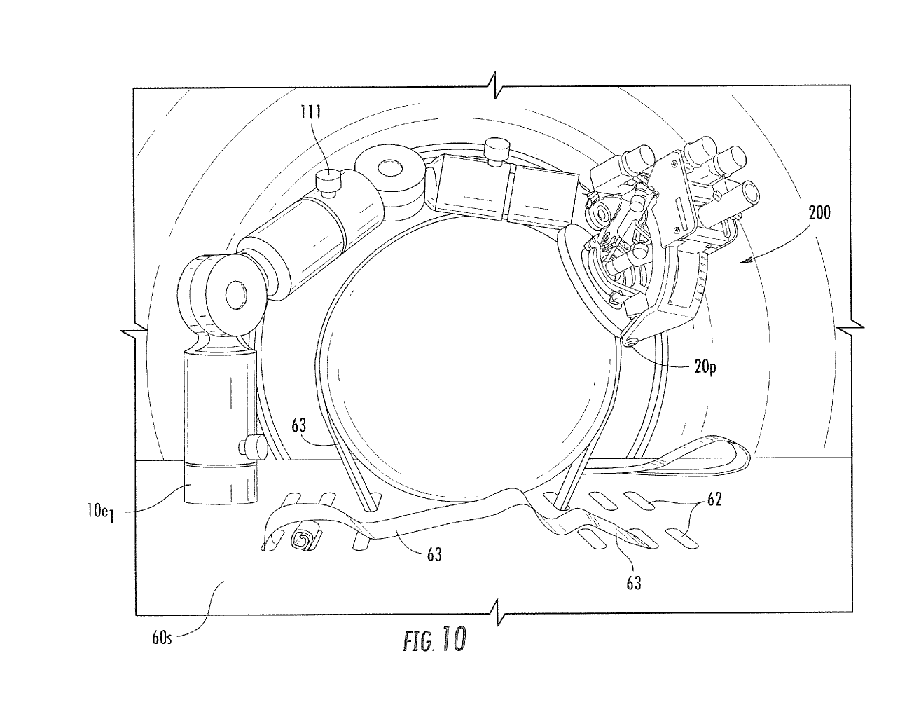

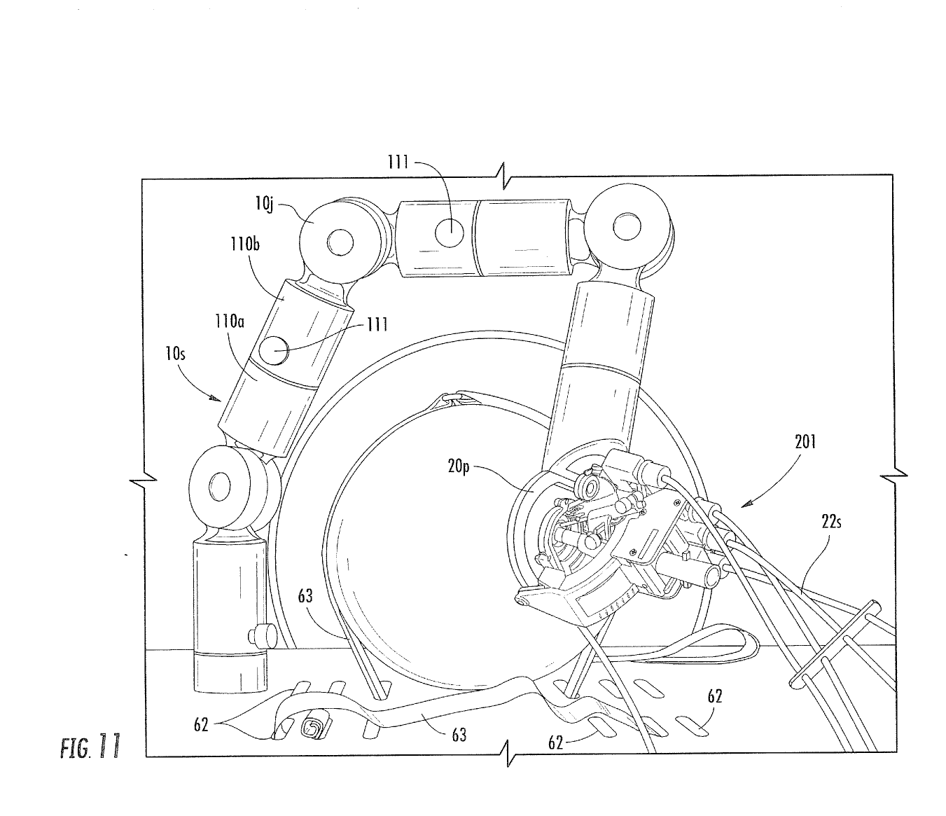

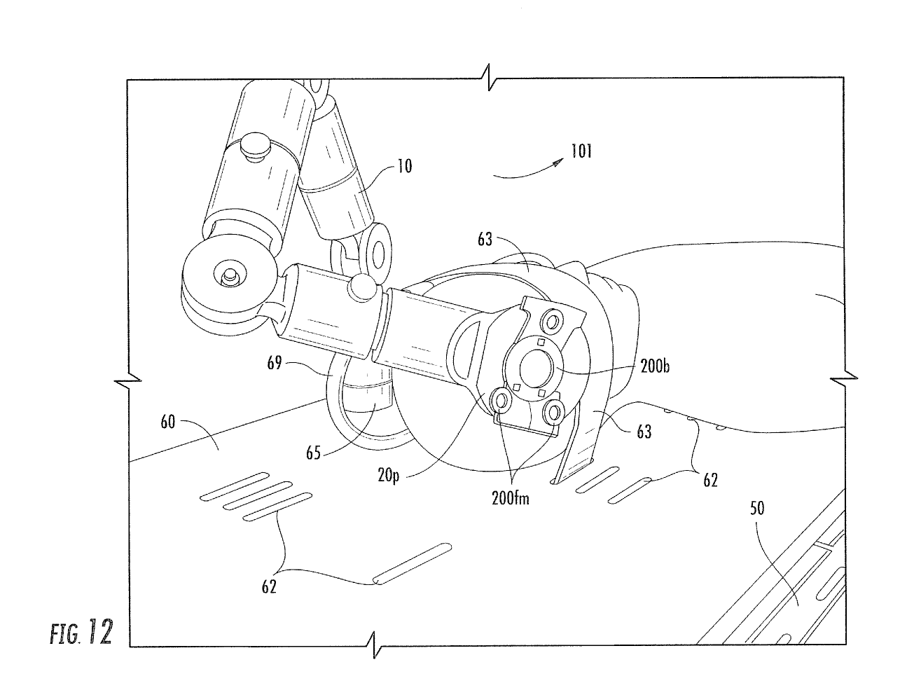

FIGS. 8 and 9 illustrate that the last segment 14 can have a single joint 10j and can be configured so that the last segment 14 defines the plate 20p at an end opposing the joint 10j. That is, the plate 20p can be an integral extension of the end of the last segment 10s.

FIGS. 8-11 also illustrate that at least some of the segments 10s can include first and second portions 110a, 110b that can move relative to each other and lock into position. The portions 110a, 110b may be able to rotate relative to each other and potentially also extend and retract relative to each other, then lock into position to define the desired arm configuration using the outer button 111 to lock or release the members to be able to move. As shown each segment 10s is connected to the next by a hinge joint 10j that provides additional degrees of freedom while providing the desired support strength and ability to lock into any number of desired configurations. Examples of suitable rotary hinges with sufficient strength for secure locking are VARILOC® hinges available from Adjustable Locking Technologies, Inc., Lathrup Village, Mich. For example, the VARILOC® plastic hinges comprise DuPont Zytel™ material with a 450 in-lb strength rating and having a 220 degree inline rotation with 10 or 45 degree incremental locking may be suitable for use. It is also contemplated that the VARILOC® stainless steel hinges may be used. See also, U.S. Pat. No. 5,689,999, the contents of which are hereby incorporated by reference as if recited in full herein. However, other hinge configurations and mechanisms may be used, including for example, gimble ball joints.

FIGS. 7-12 also illustrate that the arm 10 can move in a variety of configurations and lock the plate 20p into position such that it can be used for varying patient anatomy and target intrabody sites and fit within a magnet bore 101. FIG. 12 also illustrates that the base 60 can hold additional members 69 which may be used to help position a patient or hold head coils or other devices. As shown, an open upwardly extending rail 69 can be held on the base 60 and the patient can be strapped to the rail using a strap 63 held in slots 62 in the base 60.

In some embodiments, the attachment end 10e₁ can be configured with a suction type attachment configuration. In some embodiments, the first segment 11 can have a different configuration than the second, third and/or fourth segments 12, 13, 14. Similarly, the fourth segment 14 can have a different configuration (with the integral mounting member 20p) than the second or third segments 12, 13. The second and third segments 12, 13 can have the same configuration.

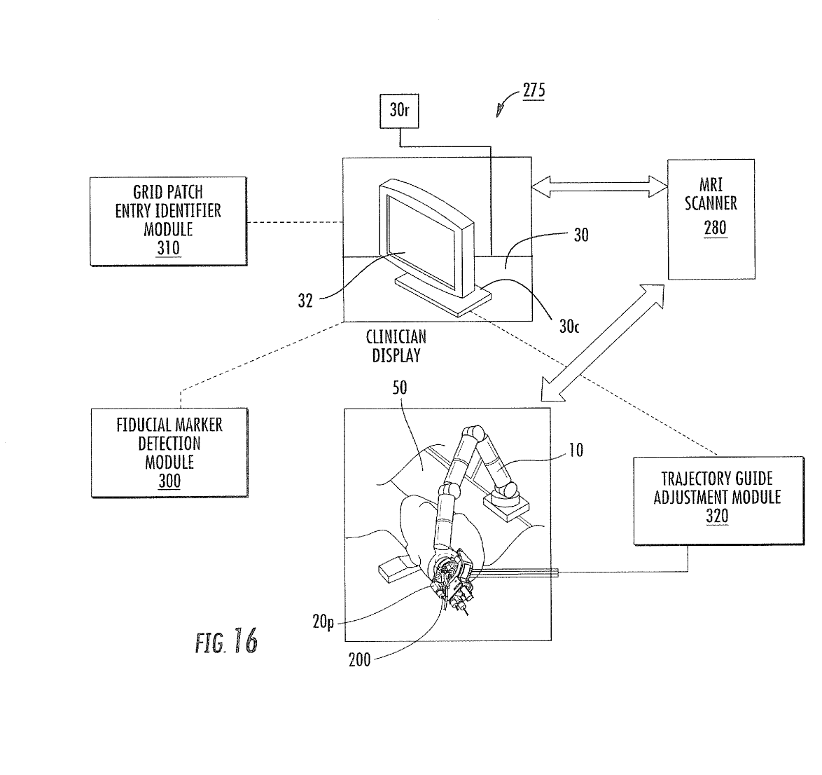

FIGS. 13 and 14 illustrate the use of the arm 10 for a torso target entry to a spinal or other target intrabody site. In the embodiment shown in FIG. 13, the base member 60 includes an integral anchor 65 and a sheet resides between the patient and the base member 60. The arm 10 is not in position while the grid 250 is placed and the target entry site is determined. FIG. 14 illustrates the arm 10 in position with the mounting member 20p over a target spinal site from an entry from the back of the patient.

FIG. 15 is a flow chart of steps that can be used to carry out methods for accessing a target intrabody surgical site associated with an MRI guided surgical procedure. An articulating arm can be mounted to be supported directly or indirectly by a scanner bed (block 80). The articulating arm can be adjusted to position a mounting member attached to the articulating arm over a desired entry site into a target intrabody surgical location (block 82). An MRI guided surgical procedure can be performed while the mounting member is held by the articulating arm over the entry site (block 88). Optionally, the arm can be held on a base member that is supported by the scanner bed (block 81).

Optionally, the method can also include placing the mounting member on the patient's body without positive retention (block 83). Optionally, the mounting member can be releasable attached, such as via a biocompatible adhesive, to a patient's body. The method may also optionally include securing a trajectory guide frame to the mounting member on an upper surface thereof (block 84); and directing a surgical tool to extend into the body through an aperture in the mounting member during the MRI guided procedure (block 85).

The mounting member can be a mounting plate with a planar lowermost surface and with an aperture extending through the plate to define a portal for surgical tools (block 87).

In some embodiments, the mounting member can include a surface coil in communication with an MRI scanner. The method can include transmitting RF signal and/or obtaining MRI signal data using the mounting member (e.g., plate) surface coil during the MRI guided procedure (block 86).

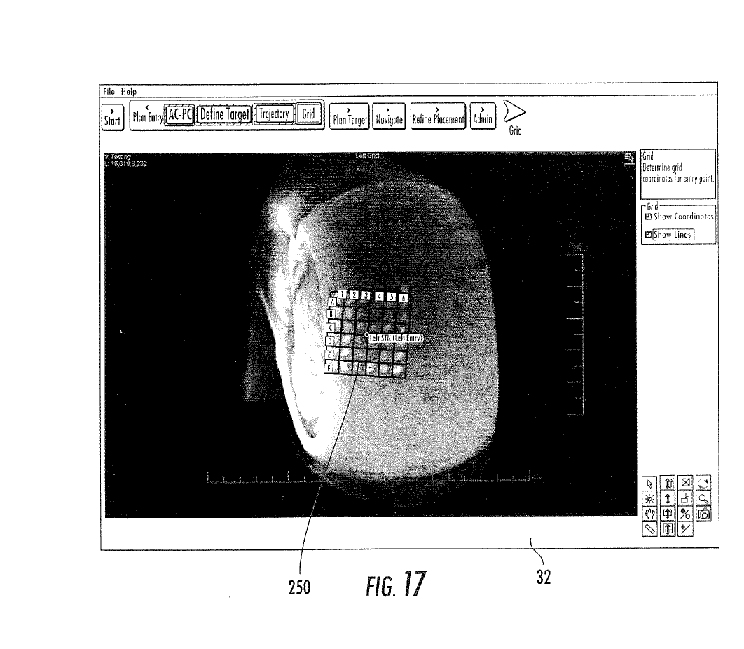

FIG. 16 is a schematic illustration of an exemplary system 275 that can be used to place the mounting member 20p over a desired entry site to help hold the trajectory guide frame 200 according to some particular embodiments of the present invention. For additional discussion of examples of planning software, see U.S. patent application Ser. No. 12/236,950, the contents of which are hereby incorporated by reference as if recited in full herein. It is noted that although shown in some figures for use in a brain procedure, this use is illustrative only and is not intended to be limiting to the use of the system/planning software for other target intrabody regions, such as those discussed above. The system 275 can include a display 32, typically at a clinician workstation 30, and an optional electronic reader 30r (for confirming the correct version of the hardware is in use). Proper operation of the system 275 (where software planning is used based on certain hardware assumptions) may require that the proper hardware having the specific predefined characteristics used by the system is used for the procedure. The system 275 can include software modules 300, 310, 320 in communication with the system circuit 30c. The term “circuit” refers to combinations of hardware and software components and can include one or more processors for carrying out the software planning.

Generally stated, particular embodiments of the invention can be configured to provide substantially automated or semi-automated and relatively easy-to-use MRI-guided systems with defined workflow steps and interactive visualizations. In some particular embodiments, the systems 275 may present workflow with discrete steps for finding target and entry point(s), localizing the entry point(s) to a physical identified grid position, guiding the alignment of the targeting frame to a planned trajectory, monitoring the insertion of a therapeutic and/or diagnostic surgical tool, and adjusting the X-Y position in cases where the placement needs to be corrected. During steps where specific MR scans are used, the system, circuit, processor or a computer module can display data requesting scan plane center and angulation to be entered at the scanner console. The workstation/circuit can passively or actively communicate with the MR scanner 280. The system 275 can also optionally be configured to use functional patient data (e.g., fiber tracks, fMRI and the like) to help plan or refine a target surgical site.

FIG. 17 illustrates an exemplary grid 250 shown overlying a patient's skull on a display 32 (without annotation lines), illustrating coordinates for selecting an entry location (shown as columns 1-6 and rows A-F) with a left STN entry location. The grid can be used at other target locations as well. Typically, a user can see the grid coordinates clearly enough that the optional overlay grid lines shown in FIG. 17 are not required in order to identify the grid elements. However, in some embodiments, as shown a UI (User Interface) will allow a user to elect to display the lines or to suppress the lines.

In some embodiments, the circuit (which includes both software and hardware aspects) can be configured to work with specific hardware (e.g., trajectory guide frame 200 and optional grid 250) with known physical attributes, fiducial markers and/or configurations. Furthermore, embodiments of the present invention may take the form of a computer program product on a computer-usable storage medium having computer-usable program code embodied in the medium. Any suitable computer readable medium may be utilized including hard disks. CD-ROMs, optical storage devices, a transmission media such as those supporting the Internet or an intranet, or other storage devices.

Computer program code for carrying out operations of the present invention may be written in an object oriented programming language such as Java®, Smalltalk or C++. However, the computer program code for carrying out operations of the present invention may also be written in conventional procedural programming languages, such as the “C” programming language. The program code may execute entirely on the user's computer, partly on the user's computer, as a stand-alone software package, partly on the user's computer and partly on another computer, local and/or remote or entirely on the other local or remote computer. In the latter scenario, the other local or remote computer may be connected to the user's computer through a local area network (LAN) or a wide area network (WAN), or the connection may be made to an external computer (for example, through the Internet using an Internet Service Provider).

The present invention is described with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer-readable memory that can direct a computer or other programmable data processing apparatus to function in a particular manner, such that the instructions stored in the computer-readable memory produce an article of manufacture including instruction means which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions/acts specified in the flowchart and/or block diagram block or blocks.

The flowcharts and block diagrams of certain of the figures herein illustrate exemplary architecture, functionality, and operation of possible implementations of embodiments of the present invention. In this regard, each block in the flow charts or block diagrams represents a module, segment, or portion of code, which comprises one or more executable instructions for implementing the specified logical function(s). It should also be noted that in some alternative implementations, the functions noted in the blocks may occur out of the order noted in the figures. For example, two blocks shown in succession may in fact be executed substantially concurrently or the blocks may sometimes be executed in the reverse order or two or more blocks may be combined, depending upon the functionality involved.

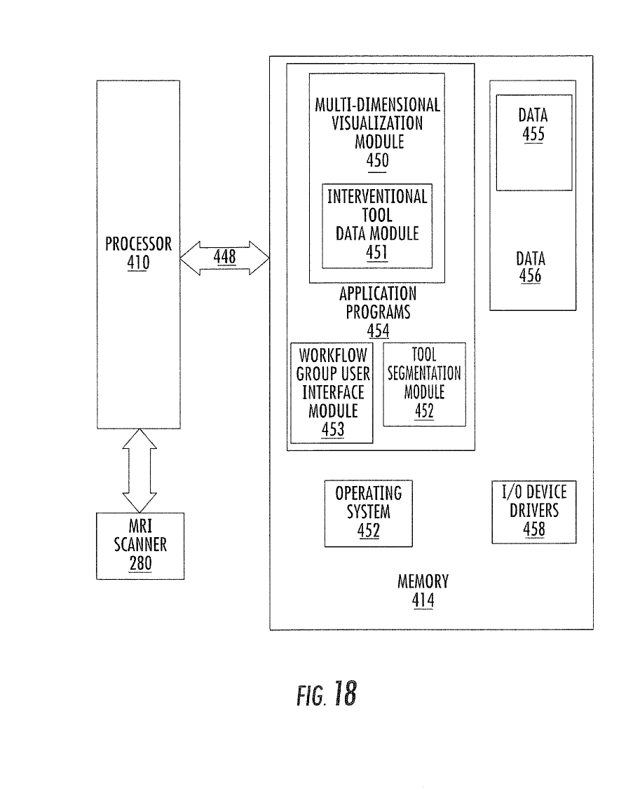

The systems 275 can include circuits and/modules that can comprise computer program code used to automatically or semi-automatically carry out operations to generate visualizations and provide output to a user to facilitate MRI-guided diagnostic and therapy procedures. FIG. 18 is a schematic illustration of a circuit or data processing system that can be used with the system 275. The circuits and/or data processing systems may be incorporated in one or more digital signal processors in any suitable device or devices. As shown in FIG. 18, the processor 410 communicates with an MRI scanner 280 and with memory 414 via an address/data bus 448. The processor 410 can be any commercially available or custom microprocessor. The memory 414 is representative of the overall hierarchy of memory devices containing the software and data used to implement the functionality of the data processing system. The memory 414 can include, but is not limited to, the following types of devices: cache, ROM, PROM, EPROM, EEPROM, flash memory, SRAM, and DRAM.

As shown in FIG. 18 illustrates that the memory 414 may include several categories of software and data used in the data processing system: the operating system 452; the application programs 454; the input/output (I/O) device drivers 458; and data 456. The data 456 can also include predefined characteristics of different surgical tools and patient image data 455. FIG. 18 also illustrates the application programs 454 can include a Visualization Module 450, Interventional Tool Data Module 451, a Tool Segmentation Module 452 (such as segmentation modules for a grid patch and/or a trajectory guide frame) and a workflow group User Interface Module 453 (that facilitates user actions and provides guidance to obtain a desired trajectory such as physical adjustments to achieve same).

As will be appreciated by those of skill in the art, the operating systems 452 may be any operating system suitable for use with a data processing system, such as OS/2, AIX, DOS, OS/390 or System390 from International Business Machines Corporation, Armonk, N.Y., Windows CE, Windows NT, Windows95, Windows98, Windows2000 or other Windows versions from Microsoft Corporation, Redmond, Wash., Unix or Linux or FreeBSD, Palm OS from Palm, Inc., Mac OS from Apple Computer, LabView, or proprietary operating systems. The I/O device drivers 458 typically include software routines accessed through the operating system 452 by the application programs 454 to communicate with devices such as I/O data port(s), data storage 456 and certain memory 414 components. The application programs 454 are illustrative of the programs that implement the various features of the data processing system and can include at least one application, which supports operations according to embodiments of the present invention. Finally, the data 456 represents the static and dynamic data used by the application programs 454, the operating system 452, the I/O device drivers 458, and other software programs that may reside in the memory 414.

While the present invention is illustrated, for example, with reference to the Modules 450-453 being application programs in FIG. 18, as will be appreciated by those of skill in the art, other configurations may also be utilized while still benefiting from the teachings of the present invention. For example, the Modules 450-453 and/or may also be incorporated into the operating system 452, the I/O device drivers 458 or other such logical division of the data processing system. Thus, the present invention should not be construed as limited to the configuration of FIG. 18 which is intended to encompass any configuration capable of carrying out the operations described herein. Further, one or more of modules, i.e., Modules 450-453 can communicate with or be incorporated totally or partially in other components, such as a workstation, an MRI scanner, an interface device. Typically, the workstation 30 will include the modules 450-453 and the MR scanner with include a module that communicates with the workstation 30 and can push image data thereto.

The I/O data port can be used to transfer information between the data processing system, the circuit 30e or workstation 30, the MRI scanner 280, and another computer system or a network (e.g., the Internet) or to other devices controlled by or in communication with the processor. These components may be conventional components such as those used in many conventional data processing systems, which may be configured in accordance with the present invention to operate as described herein.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the teachings and advantages of this invention. Accordingly, all such modifications are intended to be included within the scope of this invention as defined in the claims. The invention is defined by the following claims, with equivalents of the claims to be included therein.

That which is claimed is:

1. A medical tool, comprising:
an MRI compatible articulating arm having opposing first and second end portions, the first end portion is supported directly or indirectly by a scanner bed and the second end portion is configured to hold a device that remains in place over an entry site to a target intrabody location of a patient during an MRI guided procedure.

2. The tool of claim 1, further comprising a base member that resides on the scanner bed, wherein the first end portion of the articulating arm is attached to the base member.

3. The tool of claim 1, wherein the medical tool comprises a mounting member with an aperture extending therethrough.

4. The tool of claim 3, wherein, in use, the mounting member is configured to (a) contact a patient's body or (b) reside spaced apart from but proximate a patient's body.

5. The tool of claim 3, wherein the mounting member is a mounting plate.

6. The tool of claim 3, further comprising a trajectory guide frame held on an upper surface of the mounting member so that an intrabody device can extend through the trajectory guide frame and the mounting member aperture.

7. The tool of claim 1, wherein the articulating arm comprises at least two segments that are attached via joints that allow the arm to be configured into a plurality of different lockable orientations and shapes.

8. The tool of claim 1, wherein the articulating arm comprises a plurality of serially connected segments with at least one of the segments divided into portions that can rotate relative to each other.

9. The tool of claim 1, wherein the articulating arm comprises a non-ferromagnetic material.

10. The tool of claim 1, wherein the device held by the second end of the arm is a mounting member that comprises at least one surface coil and/or at least one gradient coil.

11. The tool of claim 2, wherein the base member comprises a surface coil.

12. The tool of claim 1, further comprising a package for holding the articulating arm in a sterile condition.

13. The tool of claim 1, wherein the device is integral to an end segment of the arm that articulates relative to an adjacent segment of the arm.

14. The tool of claim 2, wherein the base member comprises a plurality of mounting apertures residing along one long side thereof, the mounting apertures configured to receive an anchor member to hold the articulating arm.

15. The tool of claim 2, wherein the base member comprises at least one mounting aperture on a short side thereof configured to receive an anchor member associated with the articulating arm.

16. The tool of claim 2, the base member further comprising a plurality of laterally spaced apart elongate slots and at least one strap configured to enter at least two of the slots to secure a patient to the base member.

17. An MRI surgical system, comprising:
an articulating arm having a first end portion that is directly or indirectly supported by a scanner bed and having an opposing second end portion with a mounting member; and
a trajectory guide frame that is attachable to the mounting member.

18. The system of claim 17, wherein the mounting member is a mounting plate that is sterilized for medical use.

19. The system of claim 17, wherein the mounting member is a mounting plate that comprises a through-port, and wherein the mounting plate is configured to reside above or contact a patient's body so that the through-port resides over a target intrabody surgical site and allows surgical tools to extend therethrough during an MRI guided procedure.

20. The system of claim 17, further comprising a base member that resides on a scanner bed, wherein the articulating arm first end portion is attached to the base member.

21. The system of claim 20, wherein the base member comprises patient fixation means for immobilizing a target portion of a patient on the base member.

22. The system of claim 21, wherein the patient fixation means includes a plurality of slots and at least one strap that extends out through the slots and snugly around a patient for securing the patient.

23. A surgical system comprising:
a patch adapted to reside on a patient, the patch having an MRI visible grid;
an MRI compatible articulating arm with first and second opposing end portions, the first end portion being supported directly or indirectly by an MRI scanner bed, the second end portion configured to reside over a patient;
a trajectory frame held by the articulating arm; and
a processor for defining an entry site to an intrabody target in the patient using the grid.

24. The system of claim 23, further comprising a clinician workstation having a display in communication with the processor and an MRI scanner.

25. The system of claim 23, further comprising a base member that is supported by the scanner bed, and wherein the articulating arm first end portion is attached to the base member.

26. A method for accessing a target intrabody surgical site for an MRI guided surgical procedure, comprising:
providing an articulating arm so that it is supported directly or indirectly by a scanner bed;
adjusting the articulating arm to position a mounting plate attached to the articulating arm over a desired entry site into a target intrabody location; and
performing an MRI guided procedure while the mounting plate is held by the articulating arm over the intrabody location.

27. The method of claim 26, further comprising:
placing a substantially rigid base member onto a scanner bed so that it is supported by the scanner bed and can translate in and out of a magnet bore with the patient; and
attaching the articulating arm to the base member before the adjusting and performing steps.

28. The method of claim 26, further comprising;
placing the mounting plate on a patient's body;
securing a trajectory guide to the mounting plate on an upper surface of the mounting plate; and
directing a surgical tool to extend into the patient's body through an aperture in the mounting plate during the MRI guided procedure.

29. The method of claim 28, wherein the mounting plate comprises a surface coil in communication with an MRI scanner, the method further comprising transmitting RF signal and/or obtaining MRI signal data using the mounting plate surface coil during the MRI guided procedure.

30. A medical tool for an MRI guided procedure, comprising:
a plurality of discrete segments with hinge joints, at least some of the discrete segments being configured to be releasably attachable in situ to form at least a portion of a non-ferromagnetic articulating arm, wherein one of the segments includes a mounting member for holding a trajectory guide frame thereon thereby allowing a user to assemble a desired configuration of the articulating arm to accommodate a particular patient or surgical procedure.

31. A base member for an MRI guided medical procedure, the base member being non-ferromagnetic and having at least one predefined anchor location thereon for releasably attaching an articulating arm thereto, wherein the base member is configured to reside on a scanner bed and translate with the patient in and out of a magnet bore, and wherein the base member comprises patient fixation means.

32. A base member according to claim 31, wherein the patient fixation means comprises a plurality of elongate slots and at least one strap sized and configured to extend through at least two of the slots and attach to a patient to thereby immobilize a patient during a surgical procedure, and wherein the base member optionally also comprises at least one anchor for attaching to the at least one anchor location to hold the articulating arm to the base member.

33. A base member according to claim 31, wherein the at least one predefined anchor location is a plurality of spaced apart predefined anchor locations residing along at least one perimeter side thereof.
